(19) United States
(12) Patent Application Publication
(10) Pub. No.: US 2023/0248797 A1
(43) Pub. Date: Aug. 10, 2023

(54) Method for preparing medicine with Chinese yam protein extract for treating erectile dysfunction

(71) Applicant: Daqing Zhao, Changchun, Jilin (CN)

(72) Inventors: Daqing Zhao, Changchun, Jilin (CN); Siming Wang, Changchun, Jilin (CN); Meichen Liu, Changchun, Jilin (CN); Xueyuan Bai, Changchun, Jilin (CN)

(21) Appl. No.: 17/414,886

(22) PCT Filed: Aug. 28, 2020
(86) PCT No.: PCT/CN2020/111935

§ 371 Date: Jun. 16, 2021

(30) Foreign Application Priority Data
May 8, 2020 (CN) 202010381144.1

(51) Int. Cl. A61K 36/8945; A61P 15/10

(52) U.S. Cl. CPC A61K 36/8945; A61P 15/10; A61K 2236/331; A61K 2236/53

(57) ABSTRACT

The present invention relates to a method for preparing a medicine with Chinese yam protein extract for treating erectile dysfunction, which belongs to a technical field of medicine or health products. The present invention proves the improvement effect of the Chinese yam protein extract on the erectile dysfunction in a kidney-yang deficiency experimental model from in vivo and in vitro research systems at multiple levels and all aspects, wherein the Chinese yam protein extract is closely related to improvement of organ functions related to erection control, and the protective effect is different from that of phosphodiesterase type 5 inhibitors. The present invention comprises drugs and health products prepared by using the Chinese yam protein extract as a raw material, which has effects of improving and/or treating the erectile dysfunction at least on mammals.

### BACKGROUND OF THE PRESENT INVENTION

### Field of Invention

The present invention relates to a technical field of medicine or health products, and more particularly to a method for preparing a medicine with Chinese yam protein extract for treating erectile dysfunction, including medicines and health products prepared by with the Chinese yam protein extract to improve and/or treat erectile dysfunction, which are especially used, but not limited, to improve and/or treat erectile dysfunction in mammals.

### Description of Related Arts

According to traditional Chinese medicine, erectile dysfunction (ED) belongs to the categories of “impotence” and the like, which should be dialectically treated based on the overall concept. Kidney is the core of the treatment, and the deficiency of kidney is the main cause of ED. Modern medicine shows that the deficiency of kidney is caused by the disorder of various metabolic pathways in the body, which in turn leads to the ED.

Conventionally, oral phosphodiesterase type 5 (PDE5) inhibitors, such as Sildenafil, are the first-class treatments for ED. These drugs can specifically target PDE5 in the NO/cGMP pathway, thereby inhibiting the hydrolysis of cGMP, which is conducive to maintaining the relaxation of corpus cavernosa smooth muscle and exerting organ functions. However, such inhibitors are useless for ED with severe endothelial dysfunction and organ function damage (such as diabetic ED, hypertensive ED, senile ED, etc.).

Traditional Chinese medicine has the advantages of overall regulation, multiple targets, small side effects, etc. Conventionally, a variety of single Chinese medicines or compound preparations for nourishing the kidney have shown excellent effects on the treatment of ED. Chinese yam is the dried rhizome of Dioscorea opposita Thunb., which is a Chinese traditional health food a traditional Chinese medicine with medicinal and food homology. Chinese yam has the functions of nourishing the spleen and stomach, nourishing body fluid and the lungs, and nourishing the kidney. Therefore, it is used for spleen deficiency, chronic diarrhea, lung deficiency, cough and asthma, kidney-yang deficiency, spermatorrhea, vaginal discharge, frequent urination, deficiency of heat and thirst. It is reported that Chinese yam contains a variety of nutrients such as vitamins, protein, starch, free amino acids, and some minerals such as calcium, phosphorus and iron, wherein the reported functional active ingredients include polysaccharides, polyphenols, saponins, allantoin, cholesterol, ergosterol, choline, etc., providing a variety of physiological effects such as anti-oxidation, anti-tumor, lowering blood lipids, regulating intestinal flora, and enhancing body immunity. For example, Chinese yam polysaccharides have multiple functions such as enhancing humoral immunity, anti-oxidation, anti-tumor, regulating gastrointestinal tract, and lowering blood sugar; polyphenols are effective antioxidant active substances of Chinese yam, which can effectively remove free radicals in the body and lower blood lipids, and also determines the appearance and flavor of Chinese yam; the allantoin in Chinese yam can improve the skin and has a good effect on skin and tissue repair; diosgenin is distributed in the roots, stems, leaves and other parts of Chinese yam, which has the functions of anti-inflammatory, analgesic, anti-oxidation, reducing cardiovascular and cerebrovascular diseases, preventing cancer, anti-tumor, and protecting the reproductive system. Reports on the protection effect of Chinese yam extract diosgenin on the reproductive system show that Chinese yam extract diosgenin can shorten the erectile latency of mice with oligoasthenospermia induced by tripterygium glycosides, improve sperm quality and the organ coefficient of reproductive organs and immune organs, and increase the SOD activity and reduce the MDA content in testicular tissues. However, there is no follow-up study report on its mechanism of action in related articles.

Reports on the research of protein components in Chinese yam mainly focus on protein extraction, and there are few reports on activity. Published activity research reports includes in vitro antioxidant activity research, human esophageal cancer cell EC-109 inhibitory effect research, membrane protease inhibitor activity research, in vitro inhibitory effect research of α-glucosidase, research on improving immunity, research on improving the activity of mitochondrial oxidative metabolism enzymes in brain cells, applications in drugs for treating nephritis and renal hypertension, and applications in the metabolic syndrome characterized by obesity, insulin resistance, hypertension, hyperlipidemia, and fatty liver and the related damage of heart and kidney.

Up to now, there is no report on the application of Chinese yam protein extract (CYCSE) based on the kidney-yang deficiency model in China. The model establishment and evaluation system of Chinese yam for improving sexual dysfunction is simple, the material basis is not completely clear, and the research on the mechanism of action is almost blank.

### SUMMARY OF THE PRESENT INVENTION

An object of the present invention is to provide a method for preparing a medicine with Chinese yam protein extract for treating erectile dysfunction. The Chinese yam protein extract has a safe preparation process and has no toxic and side effect to the human body. It is confirmed from multiple levels and all aspects in the in vivo and in vitro research systems that improving effects of Chinese yam protein extract on kidney-yang deficiency type ED is closely related to the improvement of organ functions related to erection control, and the protective effect is different from that of PDE5 inhibitors. The present invention is particularly related, but not limited, to improving and/or treating erectile dysfunction in mammals.

Accordingly, in order to accomplish the above objects, the present invention provides:

a method for preparing a medicine for treating erectile dysfunction, comprising using Chinese yam protein extract.

The erectile dysfunction is caused by kidney-yang deficiency.

The medicine comprises drugs and health products prepared by using the Chinese yam protein extract as a raw material, which has effects of improving and/or treating the erectile dysfunction at least on mammals.

A model of the kidney-yang deficiency is based on a rat kidney-yang deficiency model induced by hydrocortisone; according to improvement of cavernosum tissue morphology, repair of cavernous smooth muscle endothelial cell functions and activation of key signal pathways for erection (NO/cGMP), the Chinese yam protein extract is proved to be effective in treating the erectile dysfunction (ED) in rats with the kidney-yang deficiency.

Effects of the Chinese yam protein extract in improving testicular function of the rats with the kidney-yang deficiency is to improve testicular tissue morphology, reduce testicular functional cell apoptosis, increase testicular leydig cell content, promote testosterone secretion, enhance sperm motility, and improve testicular fibrosis.

A preparing method of the Chinese yam protein extract (CYCSE) comprises steps of: homogenizing fresh Chinese yam with 8-20 times distilled water, and standing for 1-4 h at 4-20° C., filtering and adjusting pH of a supernatant to 1-2; filtering and collecting precipitate, adjusting pH of the precipitate to 7-8, and freeze-drying. A yield of the protein is 0.5%-3%. The protein is white or off-white loose powder with a slight smell and a light taste.

Preferably, the preparing method of the Chinese yam protein extract comprises specific steps of: homogenizing the fresh Chinese yam with 15 times the distilled water, and standing for 2 h at 4° C., filtering and adjusting the pH of the supernatant to 2.0 with HCl; filtering and collecting the precipitate, adjusting the pH of the precipitate to 7.0 with NaOH, and freeze-drying.

The Chinese yam protein extract (CYCSE) of the present invention can be made into pharmaceutically acceptable oral preparations such as oral decoctions, tablets, capsules or granules by adding commonly used pharmaceutical excipients, which can be prepared by conventional preparation methods of corresponding types of preparations.

There is no restriction on the application of the Chinese yam protein extract (CYCSE) of the present invention in preparing health products. Therefore, the extract can be added to beverages, granules, rice cakes, chocolate, candies, biscuits, chewing gum, tea, alcoholic beverages, multivitamins, and the like.

Beneficial effects of the present invention:

(1) The present invention uses Chinese yam protein extract to develop a new application in erectile dysfunction.

(2) The present invention analyzes the material basis of the prepared Chinese yam protein extract, wherein the Chinese yam protein extract contains 36% protein and 62% starch; molecular weight distribution of the protein is 32 kDa and 14.4 kDa. The protein has a small molecular weight and no peculiar smell, which is easy to absorb. Water is mainly used as a solvent during extraction process, which has no toxic and side effect to human body.

(3) Therapeutic effects of the Chinese yam protein extract of the present invention on the kidney-yang deficiency ED are systematically studied. First, based on the rat kidney-yang deficiency model induced by hydrocortisone, CYCSE is proved to be efficient of ED of the kidney-yang deficiency rats according to the improvement of the cavernosum tissue morphology, the repair of the cavernous smooth muscle endothelial cell function and the activation of the key signal pathways for erection (NO/cGMP). Moreover, the CYCSE is provided to be effective for improving testicular function according to the improvement of the testicular morphology, the reduction of the testicular function cell apoptosis, the increase of the testicular leydig cell content, the promotion of testosterone secretion, the enhancement of sperm motility and the improvement of the testicular fibrosis. The effect of CYCSE on improving testicular function is significantly better than that of sildenafil.

(4) According to the present invention, the mechanism of the CYCSE is explored in combination with in vivo and in vitro research systems. It proves that the CYCSE can induce expression of Nrf2 protein, activate Nrf2/HO-1 signal pathway, activate antioxidant defense system, and resist testicular oxidative stress. At the same time, it can improve testicular fibrosis and maintain organ function by activating TGF-β1/SMAD signal pathway. Finally, hydrogen peroxide (H₂O₂) is used to induce TM3 cells and erectile function control cells (primary corpus cavernosum smooth muscle endothelial cells) to produce oxidative stress damage, thereby further verifying recovery effects of the CYCSE on functional cell damage. In TM3 cells, the CYCSE can activate the ERK and AKT signal pathways, so as to improve H₂O₂-induced cell viability reduction, promote testosterone secretion, and increase cGMP content. The CYCSE can activate the antioxidant defense system through the Nrf2/HO-1 signal pathway, so as to reduce aggregation of reactive oxygen species (ROS). The CYCSE improves cell fibrosis degree through a TGF-β1/SMAD2/3 signal pathway, and a protective effect is significantly stronger than that of sildenafil. A Matrigel 3D culture system is used to isolate and culture mouse primary cavernous endothelial cells (MCECs), which also proves that the CYCSE can increase cell viability of MCECs induced by H₂O₂, wherein the CYCSE plays a protective role through a cascade reaction of AKT/eNOS/cGMP key pathways for erection, while sildenafil has no such effect.

### BRIEF DESCRIPTION OF THE DRAWINGS

FIG. 1 is an analysis diagram of CYCSE protein abundance;

FIG. 2 is a distribution diagram of CYCSE protein molecular weight;

FIG. 3 illustrates a control group for studying an effect of CYCSE on corpus cavernosum morphology of rats with kidney-yang deficiency (magnification: 200×);

FIG. 4 illustrates a kidney-yang deficiency group for studying the effect of the CYCSE on the corpus cavernosum morphology of the rats with the kidney-yang deficiency (magnification: 200×);

FIG. 5 illustrates a low concentration group for studying the effect of the CYCSE on the corpus cavernosum morphology of the rats with the kidney-yang deficiency (magnification: 200×);

FIG. 6 illustrates a high concentration group for studying the effect of the CYCSE on the corpus cavernosum morphology of the rats with the kidney-yang deficiency (magnification: 200×);

FIG. 7 illustrates a sildenafil group for studying the effect of the CYCSE on the corpus cavernosum morphology of the rats with the kidney-yang deficiency (magnification: 200×);

FIG. 8 illustrates MCECs isolation and culture schemes based on a Matrigel 3D culture system;

FIG. 9 illustrates an effect of the CYCSE on cell viability of MCECs, ***p<0.001 vs. model group;

FIG. 10 illustrates an effect of the CYCSE on iNOS content in kidney-yang deficiency rats, ###p<0.001 vs. control group; *p<0.05, **p<0.01, ***p<0.001 vs. model group;

FIG. 11 illustrates an effect of the CYCSE on cGMP content in the kidney-yang deficiency rats, ###p<0.001 vs. control group; ***p<0.001 vs. model group;

FIG. 12 is expressions of p-AKT/AKT and p-eNOS/eNOS of MCECs cells by Western blot;

FIG. 13 is a quantitative analysis of p-AKT/AKT expressions of the MCECs cells, ###p<0.001 vs. p<0.001 vs. control group; p<0.05, p<0.001 vs. model group;

FIG. 14 is a quantitative analysis of p-eNOS/eNOS expressions of the MCECs cells, ###p<0.001 vs. control group; **p<0.01, ***p<0.001 vs. model group;

FIG. 15 illustrates an effect of the CYCSE on cGMP content in the MCECs cells, ###p<0.001 vs. control group; *p<0.05, ***p<0.001 vs. model group;

FIG. 16 illustrates a control group for studying an effect of the CYCSE on testis morphology of the kidney-yang deficiency rats (magnification: 300×);

FIG. 17 illustrates a kidney-yang deficiency group for studying the effect of the CYCSE on the testis morphology of the kidney-yang deficiency rats (magnification: 300×);

FIG. 18 illustrates a low concentration group for studying the effect of the CYCSE on the testis morphology of the kidney-yang deficiency rats (magnification: 300×);

FIG. 19 illustrates a high concentration group for studying the effect of the CYCSE on the testis morphology of the kidney-yang deficiency rats (magnification: 300×);

FIG. 20 illustrates a sildenafil group for studying the effect of the CYCSE on the testis morphology of the kidney-yang deficiency rats (magnification: 300×);

FIG. 21 illustrates an effect of the CYCSE on apoptosis of testicular functional cells in the kidney-yang deficiency rats, ###p<0.001 vs. ***p<0.001 vs. control group; p<0.001 vs. model group;

FIG. 22 illustrates an effect of the CYCSE on 8-OHdG content in testis tissue of the kidney-yang deficiency rats, ###p<0.001 vs. control group; **p<0.01, ***p<0.001 vs. model group;

FIG. 23 illustrates an effect of the CYCSE on SOD level in the testis tissue of the kidney-yang deficiency rats, ###p<0.001 vs. control group; *p<0.05, ***p<0.001 vs. model group;

FIG. 24 illustrates an effect of the CYCSE on ROS levels in TM3 cells, ###p<0.001 vs. control group; ***p<0.001 vs. model group;

FIG. 25 is an expression of Nrf2 protein in the testis tissue of the kidney-yang deficiency rats by Western blot;

FIG. 26 is a quantitative analysis of Nrf2 protein expression in the testis tissue of the kidney-yang deficiency rats, #p<0.05 vs. control group; **p<0.01 vs. model group;

FIG. 27 illustrates an effect of the CYCSE on mRNA expressions of Nrf2 and NQO1 in the TM3 cells, #p<0.05 vs. control group; **p<0.01 vs. model group;

FIG. 28 is expressions of Nrf2 total protein, Nrf2 cytoplasmic protein, Nrf2 nuclear protein, and HO-1 protein in the TM3 cells by Western blot;

FIG. 29 is a quantitative analysis of expression levels of the Nrf2 total protein, the Nrf2 cytoplasmic protein, the Nrf2 nuclear protein, and the HO-1 protein in the TM3 cells, ###p<0.001 vs. control group; *p<0.05, ***p<0.001 vs. model group;

FIG. 30 illustrates an effect of the CYCSE on viability of the TM3 cells, ###p<0.001 vs. model group; ***p<0.001 vs. CYCSE group, wherein Sil: sildenafil; PD: ERK inhibitor PD98059; LY: AKT inhibitor LY294002;

FIG. 31 is expressions of p-ERK/ERK and p-AKT/AKT in the TM3 cells by Western blot;

FIG. 32 is a quantitative analysis of p-ERK/ERK expression in the TM3 cells, ###p<0.001 vs. model group; **p<0.01 vs. CYCSE group, wherein Sil: sildenafil; PD: ERK inhibitor PD98059; LY: AKT inhibitor LY294002;

FIG. 33 is a quantitative analysis of p-AKT/AKT expression in the TM3 cells, ###p<0.001 vs. model group; **p<0.01, ***p<0.001 vs. CYCSE group, wherein Sil: sildenafil; PD: ERK inhibitor PD98059; LY: AKT inhibitor LY294002;

FIG. 34 illustrates an effect of the CYCSE on cGMP content in the TM3 cells, ###p<0.001 vs. control group; ***p<0.001 vs. model group;

FIG. 35 illustrates an effect of the CYCSE on testosterone content in the kidney-yang deficiency rats, *p<0.05, ***p<0.001 vs. model group; #p<0.001 vs. CYCSE (80 mg/kg);

FIG. 36 illustrates an effect of the CYCSE on testosterone content of the TM3 cells, ##p<0.01 vs. model group; *p<0.05 vs. CYCSE group;

FIG. 37 is an expression of TGF-β1/SMAD2/3 signal pathway in the testis tissue of the kidney-yang deficiency rats by Western blot;

FIG. 38 is a quantitative analysis of the expression of the TGF-β1/SMAD2/3 signal pathway in the testis tissue of kidney-yang deficiency rats, ###p<0.001 vs. control group; **p<0.01, ***p<0.001 vs. model group;

FIG. 39 is a quantitative analysis of TGF-β1 fluorescence intensity in the TM3 cells, ###p<0.001 vs. control group; ***p<0.001 vs. model group;

FIG. 40 is an expression of the TGF-β1/SMAD2/3 signal pathway in the TM3 cells by Western blot;

FIG. 41 is a quantitative analysis of the expression of the TGF-β1/SMAD2/3 signal pathway in the TM3 cells, ###p<0.001 vs. control group; ***p<0.001 vs. model group.

### DETAILED DESCRIPTION OF THE PREFERRED EMBODIMENT

The present invention will be further described below with reference to embodiments, while the embodiments are not intended to be limiting in any form. The methods, equipment, and materials in the following embodiments, if not specifically stated, are all conventional in the field.

### Embodiment 1: Application of Chinese Yam Protein Extract Granule in Erectile Dysfunction

homogenizing 2.0 kg fresh Chinese yam with 14 times distilled water, and standing for 3 h at 10° C., filtering and adjusting pH of supernatant to 2 with HCl; filtering and collecting precipitate, adjusting pH of the precipitate to 7.0 with NaOH, and freeze-drying, wherein a yield of Chinese yam protein extract is 2.0%; and adding auxiliary materials (sugar, starch, dextrin, glucose, etc.) to prepare 1000 g granules.

### Embodiment 2: Application of Chinese Yam Protein Extract Oral Liquid in Erectile Dysfunction

homogenizing 3.0 kg fresh Chinese yam with 20 times distilled water, and standing for 1 h at 4° C., filtering and adjusting pH of supernatant to 1 with HCl; filtering and collecting precipitate, adjusting pH of the precipitate to 8.0 with NaOH, and freeze-drying, wherein a yield of Chinese yam protein extract is 1.8%; and adding auxiliary materials (purified water, white sugar, aspartame, xanthan gum, CMC sodium, etc.) to prepare 1.5 L oral liquid.

### Embodiment 3: Application of the Chinese Yam Protein Extract in Health Product Tableted Candy

homogenizing 2.0 kg fresh Chinese yam with 8 times distilled water, and standing for 4 h at 20° C., filtering and adjusting pH of supernatant to 1.5 with HCl; filtering and collecting precipitate, adjusting pH of the precipitate to 7.5 with NaOH, and freeze-drying, wherein a yield of Chinese yam protein extract is 2.1%; and adding tableted candy auxiliary materials (white sugar, starch, dextrin, lactose, magnesium stearate, microcrystalline cellulose, mannitol, etc.) to prepare 1.0 kg tableted candy, wherein the tableted candy can obviously play a traditional effect of Chinese yam to nourish kidney and astringent essence, and is suitable for people with kidney-yang deficiency.

### Embodiment 4: Preparation of Chinese Yam Protein Extract

homogenizing 2.0 kg fresh Chinese yam with 15 times distilled water, and standing for 2 h at 4° C., filtering and adjusting pH of supernatant to 2.0 with HCl; filtering and collecting precipitate, adjusting pH of the precipitate to 7.0 with NaOH, and freeze-drying.

According to the present invention, the application of the Chinese yam protein extract (hereinafter referred to as CYCSE) in erectile dysfunction uses specific pharmacological experiments and mechanism research methods as follows:

1. Experimental Method:

1.1 Experimental Cells and Animals

TM3 cells: purchased from Shanghai Cell Bank of the Chinese Academy of Sciences. The culture conditions are DMEM/F12+5% horse serum+2.5% fetal bovine serum+1% penicillin-streptomycin, 37° C., 5% CO₂ incubator.

SD rats: purchased from Yisi Laboratory Animal Technology Co., Ltd., SPF grade, 180-200 g. (Certificate number: SCSK (Ji) 2018-0007)

C57BL/6 mice: purchased from Yisi Laboratory Animal Technology Co., Ltd., SPF grade, 22-24 g. (Certificate number: SCSK (Ji) 2018-0007)

1.2 Composition Analysis of the Chinese Yam Protein Extract (CYCSE)

According to instructions of BCA protein detection kit (Beyotime, Shanghai, China) and starch content detection kit (Solarbio, Beijiing, China), protein and starch content in the CYCSE were determined respectively. A Coomassie Brilliant Blue method was used to detect the protein in the CYCSE, a gel imager was used to take pictures and analyze protein molecular weight distribution, an L-8900 automatic amino acid analyzer was used to determine CYCSE amino acid content, and a microplate reader was used to detect absorbance and calculate the starch content.

1.3 Construction of a Rat Model with Kidney-Yang Deficiency

SD rats were randomly divided into 5 groups with 10 rats in each group. The SD rats were fed with 25 mg/kg hydrocortisone (HCT) for 10 days to construct a kidney-yang deficiency model, and then gave different concentrations of the CYCSE for 10 days. Specific groups were as follows: control group (distilled water 20 d); kidney-yang deficiency group (HCT 10 d+distilled water 10 d); CYCSE low-concentration group (HCT 10 d+60 mg/kg CYCSE 10 d); CYCSE high-concentration group (HCT 10 d+80 mg/kg CYCSE 10 d); sildenafil group (HCT 10 d+4.4 mg/kg sildenafil 10 d). After treatment, the rats were anesthetized by intraperitoneal injection of 3% sodium pentobarbital.

1.4 Detection of Biochemical Markers

After the animal was anesthetized, blood was taken from the celiac artery and spontaneously coagulated for 25 min, and centrifuged at 2500 rpm/min for 10 min at 4° C. Supernatant was collected, namely serum samples. Contents of inducible nitric oxide synthase (iNOS), cyclic guanosine phosphate (cGMP) and testosterone in serum were detected according to instructions of rat ELISA test kit (BPRO, Shanghai, China).

1.5 Sperm Count and Vitality Check

At the end of administration, epididymis samples were collected, excess tissue was removed, and blood was washed away with saline. The samples were fixed with tweezers, so as to cut a tail side of the epididymis longitudinally. The sperm was released in a petri dish containing PBS, and a hemocytometer was used to detect number and viability of the sperm under a microscope after waiting at room temperature for 10 minutes (sperm viability is divided into 4 levels: a) move forward rapidly in a straight line, b) move forward slowly in a straight line, c) move locally, d) no move).

1.6 Histological Examination

Cavernosum tissue and testicular tissue were fixed in 4% paraformaldehyde, and then embedded in paraffin and sectioned. Testicular tissue sections were stained with hematoxylin-eosin method and Masson method respectively. Cavernosum tissue sections were stained with hematoxylin-eosin method. The testicular tissue sections were processed by immunohistochemistry, and testicular leydig cells were labeled with 3β-HSD antibody. A Nikon posture microscope was used to take photos.

1.7 ELISA Method to Detect Oxidative Stress in Testicular Tissue

Oxidative stress was evaluated with 8-hydroxy-2-deoxyguanosine (8-OHdG) and superoxide dismutase (SOD) activity. The testicular tissue in liquid nitrogen was ground into powder, homogenized in PBS and centrifuged at 2500 rpm/min for 25 min. Supernatant was collected, and 8-OHdG content and SOD activity in rat testis was detected according to instructions of rat ELISA detection kit.

1.8 TUNEL Staining

According to instructions of TUNEL in situ cell death detection kit (Roche Diagnostics, Indianapolis, USA), testicular tissue apoptosis was tested, wherein nuclei were stained with DAPI for 5 min, and pictures were took with a fluorescence microscope.

1.9 Isolation and Culture of Primary Cavernous Endothelial Cells (MCECs)

Eight-week-old C57BL/6J mice were sacrificed by cutting necks. Lower abdomen was sterilized with alcohol cotton balls, a lower abdomen incision was made with tweezers and surgical scissors, and the abdominal fascia and foreskin glands were peeled off to expose cavernosum tissue. The cavernosum tissue was separated with surgical scissors and placed in Hank's balanced salt solution containing 10% penicillin-streptomycin, and urethra and neurovascular bundles were removed under microscope to obtain clean cavernosum tissue. The obtain tissue was washed 3 times in PSB containing 10% penicillin-streptomycin, cut into small pieces of 1-2 mm³ with surgical scissors, and placed at bottoms of a pre-cooled 24-well plate with 2 pieces in each well. Each well was supplemented with 200 μL Matrigel containing 50 ng/mL VEGF-A. The MCECs were cultured in a simulated 3D environment to induce proliferation. Then the MCECs were incubate in a 37° C., 5% CO₂ incubator for 14 days until the bottoms of the wells were full. After aspirating the medium, 200 μL Dispase was added to each well for digestion in the incubator for 1 h, and an equal volume of 10 mM EDTA was added to stop the digestion. Centrifuged cell was cultured in a complete medium for the MCECs, and subsequent experiments were carried out after 2-3 generations.

1.10 Purity Identification of MCECs

Purity of the MCECs was identified by immunofluorescence method. MCECs with a density of 5*10⁴ cells/mL were processed with cell-climbing in a 6-well plate. After 24 h of attachment, the medium was removed. The MCESs were washed 3 times with pre-cooled PBS for, fixed with 4% paraformaldehyde at room temperature for 15 min, and then washed 3 times with pre-cooled PBS. 0.5% Triton X-100 containing 5% goat serum was added as a blocking and permeabilizing solution, and placed at room temperature for 30 min. Then the blocking solution was aspirated and a primary antibody was added (PECAM-1: endothelial cell marker, Desmin: smooth muscle cell marker) before incubating overnight at 4° C. and washing 3 times with pre-cooled PBS. A secondary antibody was incubate for 1 h at room temperature and washed 3 times with pre-cooled PBS. DAPI reagent was added to stain cell nucleus at room temperature for 5 min in the dark, and washed 3 times with pre-cooled PBS. The sealing solution containing fluorescence quenching agent was used for sealing, so as to observe and analyze the purity of the MCECs under a fluorescence microscope.

1.11 CCK8 Method to Detect Effects of CYCSE on Viability of TM3 Cells and MCECs

TM3 cells were placed in a 96-well plate at a density of 3×10⁴ cells/mL and divided into 6 groups: control group, H₂O₂ group, CYCSE group, ERK inhibitor (PD98059)+CYCSE group, AKT inhibitor (LY294002)+CYCSE group, and sildenafil group. After the two inhibitors were pretreated for 1 h, 62.5 μg/mL CYCSE was added and processed for 24 h, and 0.4 mM H₂O₂ was added and processed for 2 h.

The MCECs were placed on a 96-well plate at a density of 1×10⁴ cells/mL, and different concentrations of the CYCSE (31.3 μg/mL and 62.5 μg/mL) and sildenafil were added and processed for 24 h, and 0.4 mM H₂O₂ was added and processed for 2 h.

After the two kinds of cells were treated separately, CCK8 was added and incubated at 37° C. for 1 h. A microplate reader was used to measure absorbance at 450 nm and calculate the cell viability.

1.12 Determination of Testosterone Content in TM3 Cells

The TM3 cells were placed in a 6-well plate at a density of 1×10⁵ cells/mL, and pretreated with CYCSE (62.5 μg/mL) or sildenafil for 24 h and with 0.4 mM H₂O₂ for 2 h. Then cell culture supernatant was collected and centrifuged to remove precipitate. Supernatant was collected, and testosterone content in the TM3 cells was detected according to instructions of mouse ELISA detection kit. The microplate reader was used to measure absorbance at 450 nm and calculate.

1.13 Determination of Cyclic Guanosine Phosphate (cGMP) Content in TM3 Cells and MCECs

The TM3 cells were placed in a 6-well plate at a density of 1×10⁵ cells/mL, and pretreated with CYCSE (62.5 μg/mL) or sildenafil for 24 h and with 0.4 mM H₂O₂ for 2 h. The MCECs were placed in a 6-well plate at a density of 5×10⁴ cells/mL, and different concentrations of CYCSE (31.3 μg/mL and 62.5 μg/mL) were added to process for 24 h, and then 0.4 mM H₂O₂ to process for 2 h. After the two kinds of cells were processed separately, the cells were collected, washed twice with pre-cooled PBS, and resuspended in 1 mL of PBS. The cells were repeatedly frozen and thawed in liquid nitrogen for 6 times and centrifuged at 2500 r for 20 min to collect supernatant. The cGMP content in the MCECs was detected according to the instructions of the mouse ELISA detection kit, and the microplate reader was used to measure absorbance at 450 nm and calculate.

1.14 Determination of Reactive Oxygen Content in TM3 Cells

The TM3 cells were placed in a 6-well plate at a density of 1×10⁵ cells/mL, and pretreated with 62.5 μg/mL CYCSE for 24 h and with 0.4 mM H₂O₂ for 2 h. The cells were collected, washed twice with pre-cooled PBS, suspended by adding DCFH-DA buffer, and incubated for 20 min at 37° C. in the dark. After a probe was loaded, the cells were washed twice with pre-cooled PBS. Each sample was suspended by adding 300 μL PBS, and a flow cytometry is used for testing.

1.15 Expression of TGF-β1 in TM3 Cells

The expression of TGF-β1 in the TM3 cells was detected by immunofluorescence method. The TM3 cells were placed in a 6-well plate at a density of 1×10⁵ cells/mL, and pretreated with 62.5 μg/mL CYCSE for 24 h and with 0.4 mM H₂O₂ for 2 h. After that, the medium was aspirated before washing once with pre-cooled PBS, adding 4% paraformaldehyde to fix for 15 min at room temperature, and washing once with pre-cooled PBS. 0.5% Triton X-100 containing 5% goat serum was add as a blocking and permeabilizing solution, which was placed at room temperature for 30 min. After aspirating the blocking solution, TGF-β1 antibody was added to incubate overnight at 4° C. Then primary antibody was aspirated before incubating secondary antibody at room temperature for 1 h and washing twice with pre-cooled PBS. DAPI reagent was added to stain the cell nucleus before placing at room temperature for 5 min in the dark and washing twice with pre-cooled PBS. The fluorescence microscope was used to observe and take pictures.

1.16 qRT-PCR

The TM3 cells were placed in a 6-well plate at a density of 1×10⁵ cells/mL, and pretreated with 62.5 μg/mL CYCSE for 24 h and with 0.4 mM H₂O₂ for 2 h. The cells were collected into an RNase free ep tube and centrifuged at 300 g for 5 min. Then supernatant was removed and 1 mL Trizol was added before standing at room temperature for 5 min. After centrifuging at 12000 r for 5 min, precipitate was discarded. 200 μL of chloroform was then added, shook and mixed before centrifuging at 12000 r for 15 min at 4° C. Upper water phase was aspirated and an equal volume of isopropanol was added to mix. Supernatant was discarded after placing at room temperature for 10 min and centrifuging at 4° C. for 10 min at 12000 rpm. 300 μL 75% ice ethanol was added, gently shook, and centrifuged at 4° C. for 5 min at 8000 rpm before discarding supernatant. After drying at room temperature, RNA precipitate was dissolved in DEPC water and stored at −20° C. for later use. Then an agarose gel was prepared. The Marker, sample and loading buffer mixture were added to gel wells to observe with a gel imager after electrophoresis, thereby determining RNA extraction quality and RNA concentration. A reverse transcription kit was used to reverse RNA into cDNA. Primer sequences of GAPDH (SEQ ID NO:1), Nrf2 (SEQ ID NO:4) and NQO1 (SEQ ID NO:7) are shown in Table 1. SYBR Green PCR Master Mix and PCR instrument were used to detect transcription levels of Nrf2 and NQO1.

TABLE 1
qRT-PCR primer sequences

| | Primer sequence Forward | Primer sequence Reverse |
|---|---|---|
| Gene | (5′-3′) | (5′-3′) |
| GAPDH | TGTTTCCTCGTCCCGTAG | CAATCTCCACTTTGCCACT |
| | (SEQ ID NO: 2) | (SEQ ID NO: 3) |
| Nrf2 | AGCAGGACATGGAGCAAGTT | TTCTTTTTCCAGCGAGGAGA |
| | (SEQ ID NO: 5) | (SEQ ID NO: 6) |
| NQO1 | AGCCCAGATATTGTGGCCG | CCTTTCAGAATGGCTGGCAC |
| | (SEQ ID NO: 8) | (SEQ ID NO: 9) |

1.17 Western Blotting

The TM3 cells or the MCECs were placed in 6-well plates, and the CYCSE was added to process for 24 h and 0.4 mM H₂O₂ to process for 2 h. The cells were collected and washed twice with PBS, wherein supernatant was discarded. The testicular tissue in liquid nitrogen was ground into powder. 200 μL of RIPM lysis solution (containing 1% PMSF) was added to each of the above samples to lyse on ice for 30 min and centrifuge at 12000 r for 10 min at 4° C., wherein supernatant was stored at −20° C. for later use. A BCA protein content detection kit was used to determine protein content in the samples and to adjust protein concentration to the same level. An equal volume of 2× loading buffer was added to an appropriate amount of the extracted protein sample, which was mixed well, boiled for 10 min, and stored at −20° C. Each protein sample was electrophoresed by SDS-PAGE and transferred to NC membrane, blocked in 5% PBS skimmed milk powder for 1 h, and washed with PBST 3 times and 5 min for each time. PBST was removed, and primary antibody solution (GAPHD, Nrf2, HO-1, TGF-β1, SMAD2/3, ERK, p-ERK, AKT, p-AKT, eNOS, p-eNOS) was added to incubate overnight at 4° C. on a shaker. The primary antibody was then recovered, and the membrane was washed 3 times with PBST and 5 min for each time. Then the PBST was removed, and secondary antibody solution was added corresponding to each protein to incubate for 1 h at room temperature on a shaker. The secondary antibody was then aspirated and the membrane was washed 3 times with PBST and 5 min for each time. ECL chromogenic solution A was mixed with an equal amount of chromogenic solution B before evenly placed on the NC film for rendering in the dark for 1 min. A gel imager was used to render, take pictures, and analyze a gray value of a band.

1.18 Data Statistics and Analysis

All experiments were repeated three times, and results were expressed as mean±standard deviation. Graphpad Prism v6.0 software was used for one-way analysis of variance, wherein p<0.05 was considered statistically significant.

2. Results

2.1 Component Analysis of CYCSE

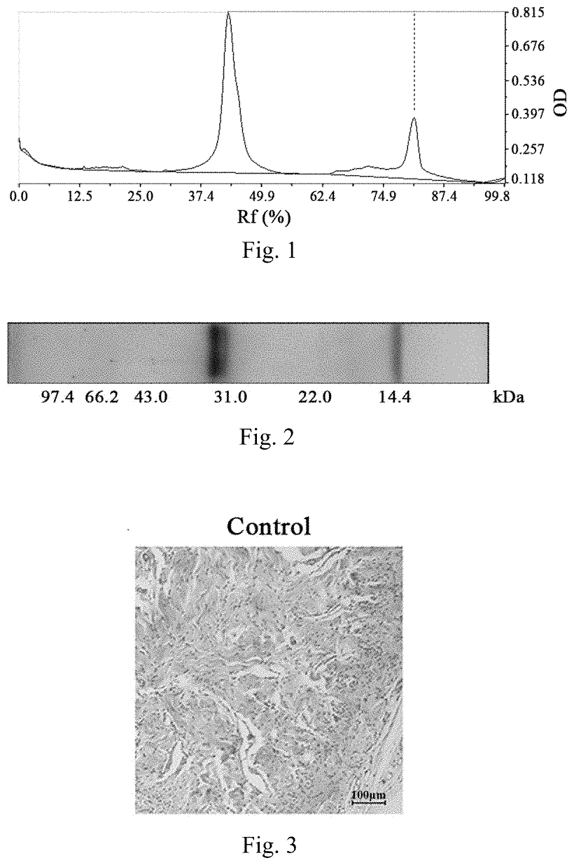

The CYCSE contains 36% protein and 62% starch. Molecular weight distribution of the protein is 32 kDa and 14.4 kDa (as shown in FIG. 1 and FIG. 2). According to an automatic amino acid analyzer which detects amino acids in the CYCSE, shows that CYCSE contains 17 kinds of amino acids, including 8 kinds required by the human body (Table 2).

TABLE 2
amino acid content in CYCSE

| | Mass fraction | | Mass fraction | Total amount |
|---|---|---|---|---|
| Name | (g · 100 g−1) | Name | (g · 100 g−1) | (%) |
| Asp | 5.137 | Ile | 1.734 | 36.01 |
| Thr | 1.432 | Leu | 3.123 | |
| Ser | 2.029 | Tyr | 1.291 | |
| Glu | 5.391 | Phe | 2.505 | |
| Gly | 1.581 | Lys | 2.070 | |
| Ala | 1.715 | Pro | 1.567 | |
| Cys | 0.187 | His | 0.897 | |
| Val | 2.077 | Arg | 3.178 | |
| Met | 0.096 | | | |

2.2 Improvement of Erectile Function and Maintenance of Related Organs in Kidney-Yang Deficiency Rats by CYCSE

Hydrocortisone (HCT) is used to establish a rat kidney-yang deficiency model, so as to confirm in vivo that the CYCSE has a potential therapeutic effect on ED in kidney-yang deficiency rats, and it is closely related to the improvement of organ functions.

2.2.1 Impact on Routine Indicators

Weight change of animal organ is one of the important biological characteristic indexes, which can explain its function strength to a certain extent. The research results indicate that compared with the rats in the control group, the rats in the model group had reduced activity, chills, bunching up, dull coat color, unresponsiveness, and significantly reduced body weight, testicular weight, and epididymal weight. After CYCSE intervention, the rat activities can be recovered, the coat color can be restored, and the body weight, testis and epididymis weights can be significantly increased compared with the model group, which are dose-dependent (as shown in Table 3).

TABLE 3
body weight and organ weight of rat

| | Body | Testicular | Epididymal |
|---|---|---|---|
| | weight (g) | weight (g) | weight (g) |
| Control | 358.13 ± 10.09 | 3.12 ± 0.02 | 0.87 ± 0.04 |
| HCT | 295.04 ± 3.76^{###} | 2.66 ± 0.05^{###} | 0.77 ± 0.02^{###} |
| CYCSE | 330.16 ± 6.46*** | 2.93 ± 0.13* | 0.81 ± 0.02** |
| (60 mg/kg) | | | |
| CYCSE | 343.32 ± 11.59*** | 3.08 ± 0.14** | 0.87 ± 0.01*** |
| (80 mg/kg) | | | |
| Sildenafil | 329.37 ± 10.82*** | 3.03 ± 0.06** | 0.85 ± 0.01*** |

^{###}P < 0.001 vs. control group;
*P < 0.05,
**P < 0.01,
***P < 0.001 vs. model group

2.2.2 Effect on Erectile Function of Kidney-Yang Deficiency Rats

The Chinese yam protein extract (CYCSE) has a therapeutic effect on erectile dysfunction in rats with kidney-yang deficiency induced by hydrocortisone. Experimental results show that such effect is realized by improving cavernosum tissue morphology, repairing functions of cavernous smooth muscle endothelial cells, and activating a key signal pathway for erection (NO/cGMP).

2.2.2.1 Improvement of Corpus Cavernosum Morphology

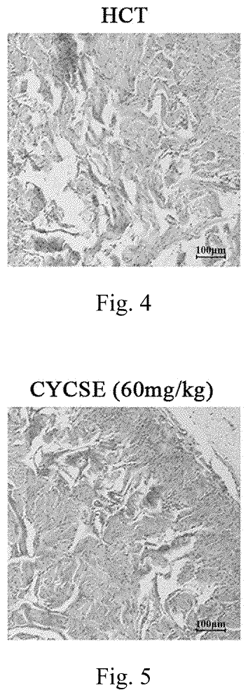

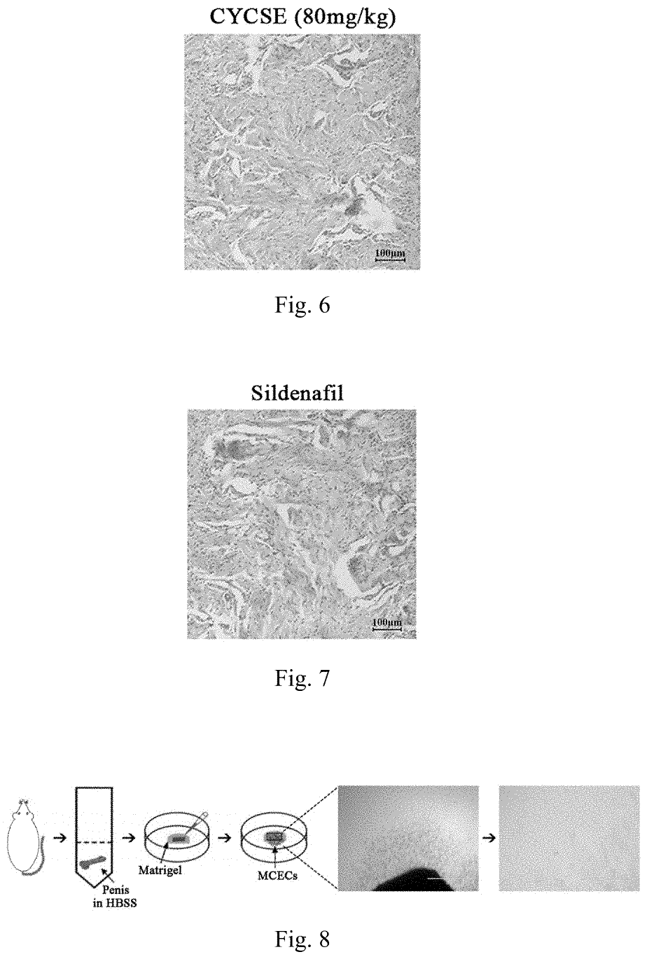

The cavernosum plays a decisive role in penile erection. Results of tissue morphology study show that compared with the control group, the cavernosum smooth muscle layer is thin and the cavernous sinus is disordered and discontinuous in the model group, suggesting that the physiological function of the cavernosum has changed and cannot achieve normal erectile function. After CYCSE intervention, discontinuous arrangement of smooth muscle and endothelial cells in the cavernosum tissue, as well as disorder of interstitial cells are significantly improved (as shown in FIGS. 3-7).

2.2.2.2 Function Repair of Cavernous Smooth Muscle Endothelial Cells of Penis

The cavernous smooth muscle endothelial cells are the key cells to control erection. This research optimizes the choice of models. In previous studies, human umbilical vein endothelial cells (HUVECs) are usually used. Such cell model cannot accurately simulate a microvascular environment of cavernous endothelial cells. The primary cavernous endothelial cells (MCECs), located on an inner surface of the cavernosum, are one of the most important cells to maintain the function of the cavernosum, and are the best choice for studying ED endothelial function. When comes to cell separation methods, the enzyme separation method is most common choice, but the operation is cumbersome, the purity is low, the cell damage is strong, and the repeatability is poor. The Matrigel 3D culture system is a novel non-enzymatic separation method that can simulate the three-dimensional environment of cell growth in the body, allowing MCECs to directly contact with growth factors, so as to induce them to crawl out of the tissue, ensuring the original morphology and functional characteristics of the MCECs. Moreover, the operation is time-saving, the separation purity is high, and the repeatability is good, which make it the best separation scheme for studying the function of ED endothelium. FIG. 8 shows the isolation and culture scheme of the MCECs based on the Matrigel 3D culture system.

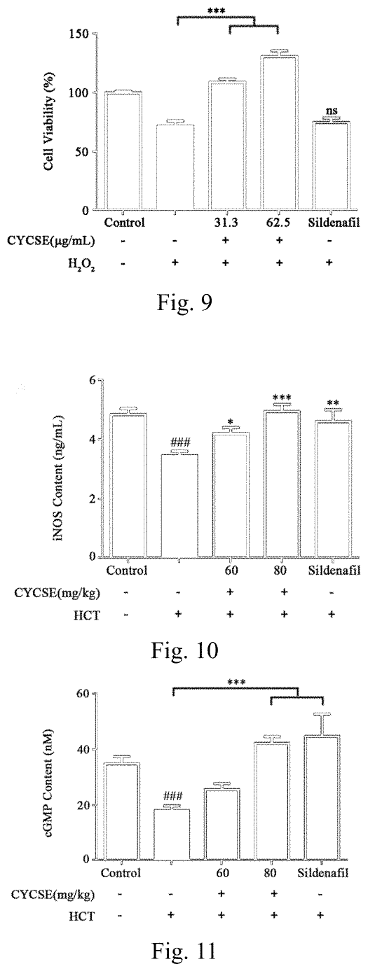

Accordingly, the Matrigel 3D culture system is used to isolate MCECs for studying effects of the CYCSE on cell viability of H₂O₂ damaged MCECs. The CCK8 method is used to investigate the cell viability. It can be seen from FIG. 9 that the CYCSE significantly increases the cell viability of oxidation-damaged MCECs cells, which is dose-dependent. Sildenafil has no salvage effect. The in vitro model further confirms that the CYCSE can improve ED.

2.2.2.3 Activation of Key Signal Pathway for Erection (NO/cGMP)

In order to further determine a physiological correlation between the CYCSE and the erectile function of kidney-yang deficiency rats, the key signal pathway (NO/cGMP signal pathway) that controls erectile function is studied. A non-adrenergic non-cholinergic (NANC) mechanism is a main mechanism that regulates relaxation of the vascular smooth muscle of the cavernosum of the penis. Among them, nitric oxide (NO) is considered to be a main neurotransmitter, and the NO/cGMP pathway plays an important regulatory role in the process of penile erection. NANC nerve endings, vascular endothelial cells and penile cavernous endothelial cells release NO under catalysis of nitric oxide synthase (NOS), wherein NO rapidly diffuses into smooth muscle cells through cell membrane, and activates guanylate cyclase to increase synthesis of cyclic guanosine phosphate (cGMP), thereby inducing penile erection through a series of cascade reactions. Therefore, NOS and cGMP are core components of the NO/cGMP signal pathway, and their content can be used to evaluate penile erectile function.

According to in vivo research results, contents of inducible nitric oxide synthase (iNOS) and cGMP in the corpus cavernosum of the model group are significantly reduced, while the contents of iNOS and cGMP are significantly increased after CYCSE intervention. There is no significant difference between the sildenafil group and the high concentration group (as shown in FIG. 10 and FIG. 11).

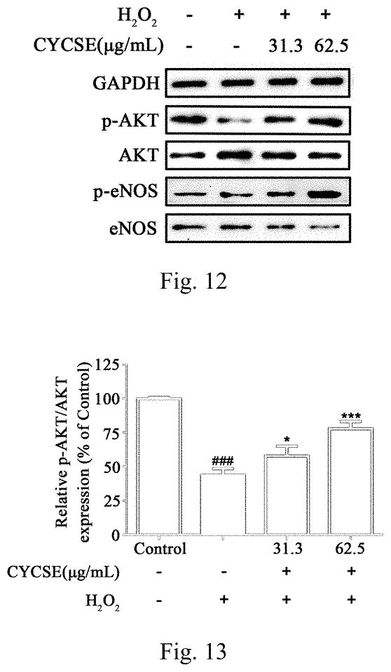

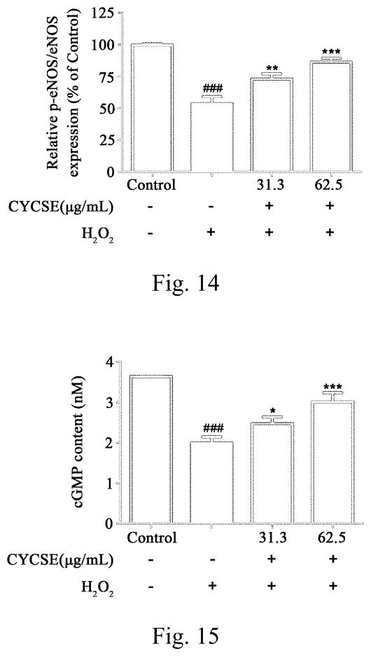

In cavernous endothelial cells, endothelial nitric oxide synthase (eNOS) is activated under the action of calcium ions to regulate the NO/cGMP pathway and promote penile erection. However, during penile erection, calcium dependence of eNOS is very short-lived. An AKT pathway can directly cause phosphorylation of eNOS, reduce demand for calcium ions, and further promote production of NO to perform organ functions. According to results of in vitro studies, the CYCSE can promote expression of phosphorylated AKT and eNOS in oxidation-damaged MCECs (as shown in FIGS. 12-14), increase the content of cGMP (FIG. 15), promote occurrence of AKT/eNOS/cGMP cascade, and enhance erectile function.

2.2.3 Effect on Testicular Function of Kidney-Yang Deficiency Rats

The effects of the Chinese yam protein extract (CYCSE) on improving the testicular function of kidney-yang deficiency rats are determined by improving testicular morphology, reducing testicular functional cell apoptosis, increasing testicular leydig cell content, promoting testosterone secretion, enhancing sperm motility, and improving testicular fibrosis.

2.2.3.1 Improvement of Testicular Tissue Morphology

Impairment of testicular function is a core factor that induces ED. Therefore, in order to explore whether the improvement effect of the CYCSE on ED of the kidney-yang deficiency rats is related to rescue of the testicular function, the testicular tissue morphology is studied first.

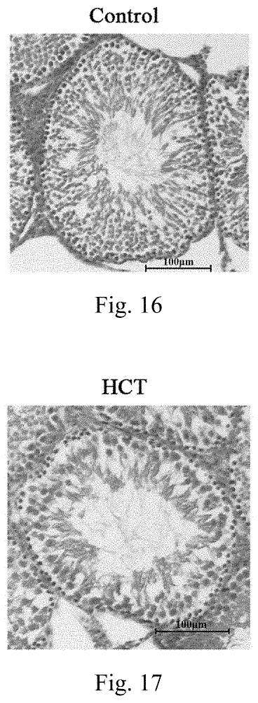

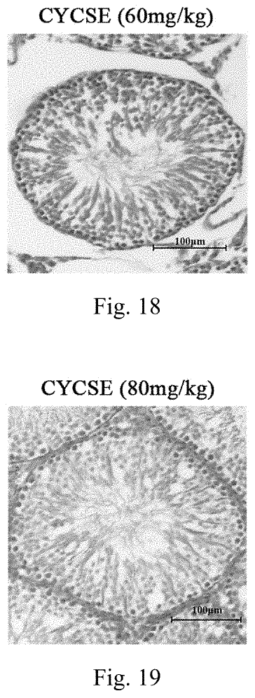

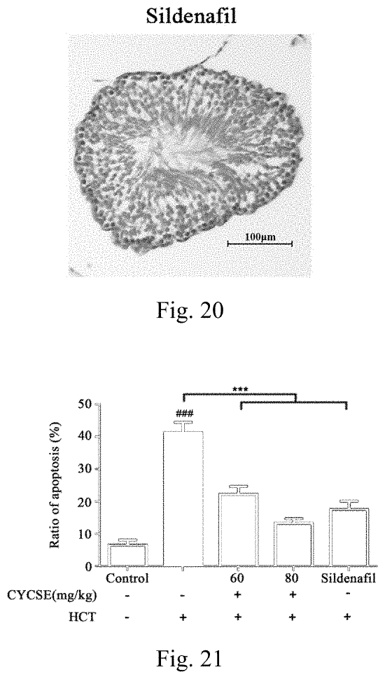

Morphological observation of rat testis is carried out with HE staining method. Compared with the control group, testicular seminiferous tubules of the model group are atrophied, germ cell layer is reduced, and testis tissue is damaged. The CYCSE can effectively improve atrophy of the testicular seminiferous tubules of the rats in the model group and increase the number of germ cell layers, which is dose-dependent (as shown in FIGS. 16-20 and Table 4).

TABLE 4
testicular health parameters

| | Diameter of seminiferous | Germinal cell layer |
|---|---|---|
| | tubules (μm) | thickness (μm) |
| Control | 359.68 ± 10.22 | 80.25 ± 4.74 |
| HCT | 296.20 ± 2.19^{##} | 40.51 ± 1.82^{##} |
| CYCSE (60 mg/kg) | 326.65 ± 0.73* | 68.59 ± 0.39 |
| CYCSE (80 mg/kg) | 352.19 ± 7.67** | 76.57 ± 7.30* |
| Sildenafil | 357.61 ± 8.03** | 72.50 ± 5.47** |

^{##}P < 0.01 vs. control group;
*P < 0.05,
**P < 0.01 vs. model group

2.2.3.2 Apoptosis Reduce of Testicular Function Cells

Cell production and maturation are closely related to cell apoptosis. Apoptosis within a certain range has positive physiological significance to the body, but excessive apoptosis will significantly reduce secretion of testosterone, leading to increased spermatogenic cell apoptosis and even infertility. The TUNEL method is used to characterize the apoptosis of functional cells in the testis, and ImagePro software is used to analyze images. It can be seen from FIG. 21 that compared with the control group, apoptotic cells in the testis tissue of kidney-yang deficiency rats are increased in the model group. After the CYCSE intervention, the apoptotic cells in the two concentration groups are reduced by about 2-3 times compared with the model group.

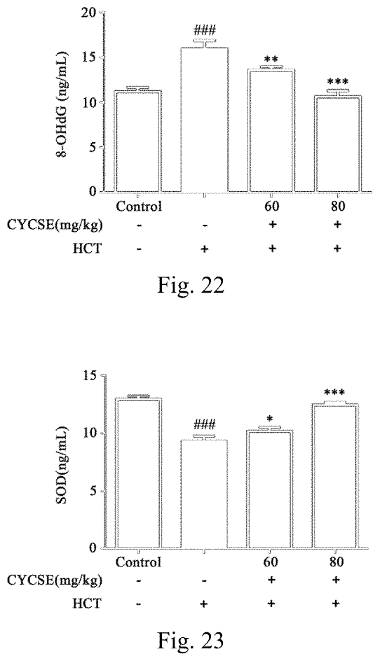

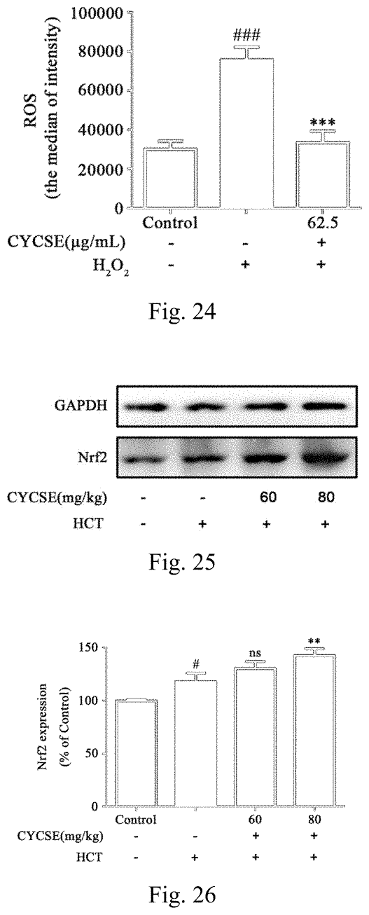

Excessive production of reactive oxygen species (ROS) is closely related to excessive cell apoptosis and is a core factor in inducing cell apoptosis. Under a variety of endogenous or exogenous stimuli, generation or removal rate of ROS is destroyed, leading to excessive ROS accumulation and destroying redox balance in the body, which finally triggers oxidative stress. Hydrocortisone is a glucocorticoid, which can stimulate oxidative stress and induce excessive cell apoptosis. Therefore, the present invention further uses ROS, superoxide dismutase (SOD) and 8-hydroxy-2-deoxyguanosine (8-OHdG) to evaluate the repair effects of the CYCSE on the testicular tissue and testicular interstitial TM3 cell oxidative stress of oxidation-damaged kidney-yang deficiency rats induced by hydrogen peroxide (H₂O₂). Results show that 8-OHdG content and SOD level of the rats in the model group deviated from a normal level, indicating that the testis is in a state of oxidative stress. After the CYCSE intervention, the 8-OHdG content is significantly reduced, and the SOD level is significantly increased (as shown in FIGS. 22-23). A flow cytometry is used to detect cellular ROS levels. It can be seen from FIG. 24 that the CYCSE can significantly reduce the excessive release of ROS in the TM3 cells caused by oxidation damage.

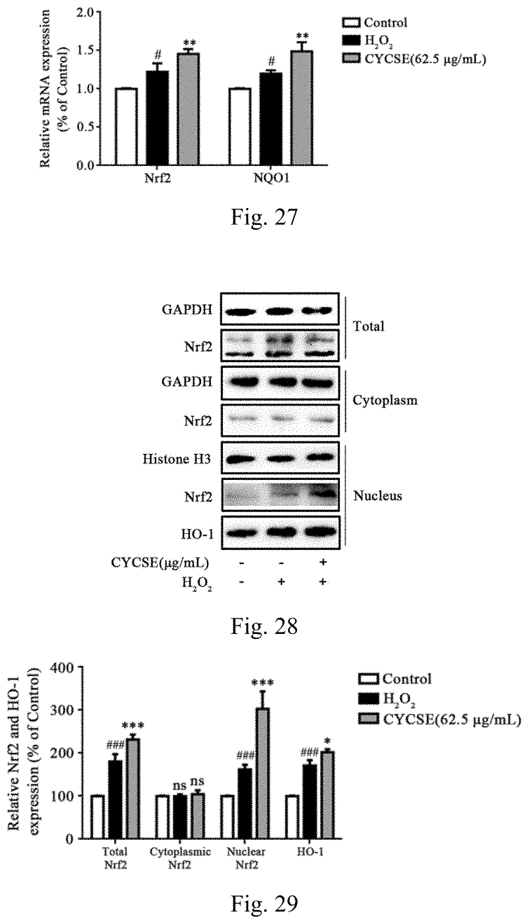

Nrf2 is an important transcription factor which regulates the oxidative stress response of cells, and it is also a central regulator that maintains the intracellular redox homeostasis. Nrf2 regulates expressions of a series of antioxidant factors (such as HO-1, NQO1), reduces cell damage caused by reactive oxygen species and electrophiles, keeps cells in a stable state, and maintains dynamic balance of redox homeostasis. According to the present invention, Western blot is used to detect the expression of Nrf2 protein in the testicular tissues, wherein the expression of Nrf2 protein in the model group is significantly higher than that of the control group. With CYCSE protection, the expression of Nrf2 in the damaged testicular tissue can be further increased, thereby activating an antioxidant defense system (as shown in FIGS. 25-26). Protein and transcription levels of oxidative stress key regulatory target Nrf2 and downstream anti-oxidative stress factors in the TM3 cells are detected and analyzed. Results show that the CYCSE can significantly increase transcription and protein expression of Nrf2, and promote transfer of Nrf2 to the nucleus, thereby increasing expressions of downstream factors HO-1 and NQO1 and activating the cellular antioxidant defense system (as shown in FIGS. 27-29).

2.2.3.3 Increase the Content of Testicular Leydig Cells

Leydig cells are endocrine gonadal epithelial cells, which are the most important cells producing testosterone in male animals and one of the most important functional cells in the testicular tissue. In immunohistochemical experiment, 3β-HSD is used to specifically label the testicular leydig cells, which indicates that compared with the control group, the 3β-HSD immunopositive cells in the testis tissue of the model group are significantly reduced. After the CYCSE intervention with different concentrations, immunostaining intensity of the testicular leydig cells is significantly stronger than that of the model group, and a effect of the high-concentration group is stronger than that of sildenafil (as shown in Table 5).

TABLE 5
semi-quantitative analysis of testicular leydig cells

| | Immunoexpression of 3β-HSD |
|---|---|
| Control | +++^{Δ} |
| HCT | − |
| CYCSE (60 mg/kg) | + |
| CYCSE (80 mg/kg) | ++ |
| Sildenafil | + |

^{Δ}immunostaining intensity is scored with a simplified scale, ranging from negative (−) to weakly positive (+) to strong positive (+++).

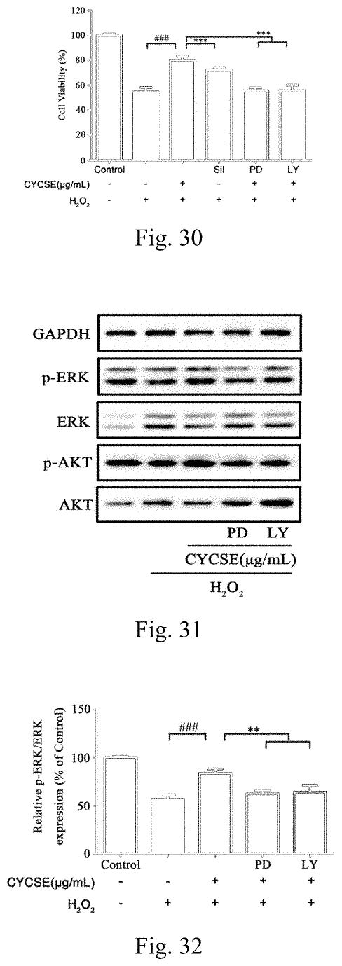

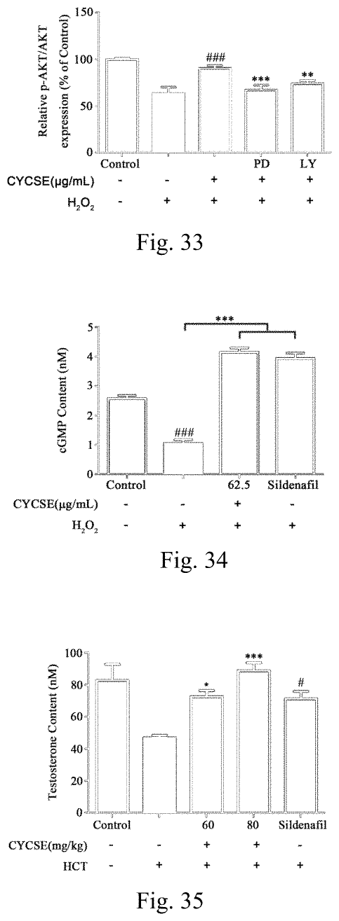

The CCK8 method is further used to investigate the viability of testicular interstitial TM3 cells in vitro. It can be seen that the CYCSE can significantly increase the cell viability of the oxidation-damaged TM3 cells and promote cell proliferation, whose effect is significantly stronger than that of sildenafil (see FIG. 30). Conventional research indicates that ERK and AKT signal pathways are the core control pathways that regulate cell proliferation. In order to find out whether the effect of the CYCSE on promoting the proliferation of the TM3 cells is related to the ERK and AKT signal pathways, the present invention adopts an ERK inhibitor (PD98059) and an AKT inhibitor (LY294002). FIG. 30 shows that the two inhibitors can significantly block the protective effect of the CYCSE on the TM3 cells. At the same time, Western blot analysis indicates that expressions of p-ERK/ERK and p-AKT/AKT are increased significantly after the oxidation-damaged TM3 cells are protected by the CYCSE, while such increase is significantly down-regulated under with the two inhibitors. It shows that the rescue effect of the CYCSE on the viability of the oxidation-damaged TM3 cells is achieved by activating the ERK and AKT signaling pathways (see FIGS. 31-33). In addition, in the oxidation-damaged TM3 cells, CYCSE protection can significantly increase the cGMP content induced by H₂O₂ (as shown in FIG. 34).

2.2.3.4 Promotion of Testosterone Secretion

Testosterone is a very important male hormone, mainly secreted by the testicular leydig cells, and is an important indicator for evaluating organ function. The CYCSE can significantly increase serum testosterone content in the kidney-yang deficiency rats, which is consistent with the significant increase in testicular leydig cell content. The effect of the high concentration group is significantly stronger than that of sildenafil (as shown in FIG. 35).

The testosterone content secreted in the TM3 cells is further detected. Referring to FIG. 36, testosterone secretion ability of the TM3 cells is reduced due to the induction of H₂O₂. The CYCSE protection can significantly increase the secretion of testosterone, whose effect is significantly stronger than that of sildenafil.

2.2.3.5 Enhancement of Sperm Motility

Sperm motility is another important indicator for evaluating the testicular function. It can be seen from Table 6 that the number of sperm in the model group is significantly reduced, and the percentages of sperm motility grades a and a+b are reduced by about half compared with the control group. After CYCSE treatment, the number of sperm and the percentage of sperm motility grades a and a+b are increased significantly, which are dose-dependent. Furthermore, three indexes of the CYCSE high concentration group are significantly higher than those of the sildenafil group.

TABLE 6
sperm number and motility

| | Sperm number | Sperm motility (%) | |
|---|---|---|---|
| | (*10⁶/mL) | a | a + b |
| Control | 6.49 ± 0.56 | 26.33 ± 2.49 | 58.33 ± 3.68 |
| HCT | 3.79 ± 0.53^{##} | 10.33 ± 2.05^{##} | 27.67 ± 2.05^{##} |
| CYCSE | 5.12 ± 0.28* | 18.67 ± 2.49* | 42.00 ± 2.94** |
| (60 mg/kg) | | | |
| CYCSE | 6.18 ± 0.52** | 25.00 ± 1.63** | 50.33 ± 2.06*** |
| (80 mg/kg) | | | |
| Sildenafil | 4.63 ± 0.56^{Δ} | 17.67 ± 2.05^{Δ} | 37.67 ± 2.05^{ΔΔ} |

^{##}P < 0.01 vs. control group;
*P < 0.05,
**P < 0.01,
***P < 0.001 vs. model group;
^{Δ}P < 0.05,
^{ΔΔ}P < 0.01 vs. CYCSE (80 mg/kg)

2.2.3.6 Improvement of Testicular Fibrosis

Testicular fibrosis is an important factor for disturbing spermatogenesis environment and destroying spermatogenesis, which is a key to testicular function damage. First, Masson staining is used to analyze expression of collagenous fibers in the testicular tissues (as shown in Table 7). In the rats of the model group, a large amount of collagen is leaked into the interstitial tissue, and the testicular tissue is fibrotic. After the CYCSE treatment, collagenous fibers are decreased significantly with concentration increase of administration.

TABLE 7
Semi-quantitative analysis of testicular fibrosis

| | Immunoexpression of collagenous fibers |
|---|---|
| Control | −^{Δ} |
| HCT | +++ |
| CYCSE (60 mg/kg) | ++ |
| CYCSE (80 mg/kg) | + |

^{Δ}immunostaining intensity is scored with a simplified scale, ranging from negative (−) to weakly positive (+) to strong positive (+++).

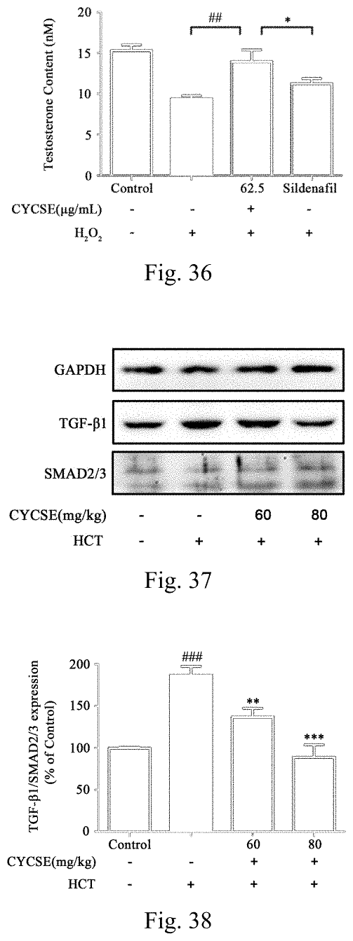

TGF-β1 provides signal stimulation during tissue repair and fibrosis. By initiating intracellular signal cascade, TGF-β1 activates SMAD2/3 to form a complex, enters the nucleus, and regulates excessive proliferation of collagen, thereby causing fibrosis. Western blot results show that the CYCSE can reduce fibrosis degree of the testis tissue in kidney-yang deficiency rats by inhibiting TGF-β1/SMAD2/3 signal pathway (as shown in FIGS. 37-38).

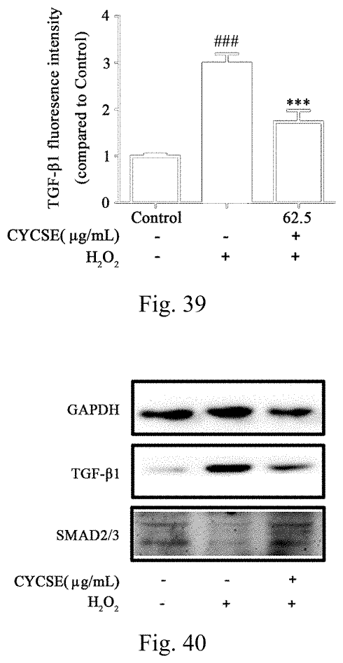

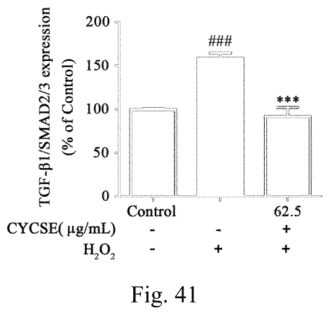

Immunofluorescence is used to specifically label TGF-β1 in the TM3 cells and analyze an expression level. Compared with the control group, fluorescence intensity of TGF-β1 in the damaged TM3 cells is significantly increased, and overexpression of TGF-β1 is significantly inhibited after the CYCSE protection (as shown in FIG. 39). Western blot results further show that CYCSE can down-regulate the expression of the TGF-β1/SMAD2/3 signaling pathway in the oxidation damaged TM3 cells, thereby reducing the fibrosis degree of the damaged TM3 cells (as shown in FIG. 40-41).

What is claimed is:

1. A method for preparing a medicine for treating erectile dysfunction, comprising using Chinese yam protein extract.

2. The method, as recited in claim 1, wherein the erectile dysfunction is caused by kidney-yang deficiency.

3. The method, as recited in claim 1, wherein the medicine comprises drugs and health products prepared by using the Chinese yam protein extract as a raw material, which has effects of improving and/or treating the erectile dysfunction at least on mammals.

4. The method, as recited in claim 2, wherein a model of the kidney-yang deficiency is based on a rat kidney-yang deficiency model induced by hydrocortisone; according to improvement of cavernosum tissue morphology, repair of cavernous smooth muscle endothelial cell functions and activation of key signal pathways for erection (NO/cGMP), the Chinese yam protein extract is proved to be effective in treating the erectile dysfunction (ED) in rats with the kidney-yang deficiency.

5. The method, as recited in claim 4, wherein effects of the Chinese yam protein extract in improving testicular function of the rats with the kidney-yang deficiency is to improve testicular tissue morphology, reduce testicular functional cell apoptosis, increase testicular leydig cell content, promote testosterone secretion, enhance sperm motility, and improve testicular fibrosis.

6. The method, as recited in claim 1, wherein a preparing method of the Chinese yam protein extract comprises steps of: homogenizing fresh Chinese yam with 8-20 times distilled water, and standing for 1-4 h at 4-20° C., filtering and adjusting pH of a supernatant to 1-2; filtering and collecting precipitate, adjusting pH of the precipitate to 7-8, and freeze-drying.

7. The method, as recited in claim 6, wherein the preparing method of the Chinese yam protein extract comprises specific steps of: homogenizing the fresh Chinese yam with 15 times the distilled water, and standing for 2 h at 4° C., filtering and adjusting the pH of the supernatant to 2.0 with HCl; filtering and collecting the precipitate, adjusting the pH of the precipitate to 7.0 with NaOH, and freeze-drying.


SEQUENCE LISTING (9 sequences)

SEQ ID NO: 1 - DNA, 1296 bp, Mus musculus
aacccttaagagggatgctgcccttaccccggggtcccagcttaggttcatcaggtaaactcaggagagtgtttcctcgtcccgtagacaaaatggtgaaggtcggtgtgaacggatttggccgtattgggcgcctggtcaccagggctgccatttgcagtggcaaagtggagattgttgccatcaacgaccccttcattgacctcaactacatggtctacatgttccagtatgactccactcacggcaaattcaacggcacagtcaaggccgagaatgggaagcttgtcatcaacgggaagcccatcaccatcttccaggagcgagaccccactaacatcaaatggggtgaggccggtgctgagtatgtcgtggagtctactggtgtcttcaccaccatggagaaggccggggcccacttgaagggtggagccaaaagggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatgaccacagtccatgccatcactgccacccagaagactgtggatggcccctctggaaagctgtggcgtgatggccgtggggctgcccagaacatcatccctgcatccactggtgctgccaaggctgtgggcaaggtcatcccagagctgaacgggaagctcactggcatggccttccgtgttcctacccccaatgtgtccgtcgtggatctgacgtgccgcctggagaaacctgccaagtatgatgacatcaagaaggtggtgaagcaggcatctgagggcccactgaagggcatcttgggctacactgaggaccaggttgtctcctgcgacttcaacagcaactcccactcttccaccttcgatgccggggctggcattgctctcaatgacaactttgtcaagctcatttcctggtatgacaatgaatacggctacagcaacagggtggtggacctcatggcctacatggcctccaaggagtaagaaaccctggaccacccaccccagcaaggacactgagcaagagaggccctatcccaactcggcccccaacactgagcatctccctcacaatttccatcccagacccccataataacaggaggggcctagggagccctccctactctcttgaataccatcaataaagttcgctgcacccacaaaaaaaaaaaaaaaaaaaaaa

SEQ ID NO: 2 - protein, 18 aa, Artificial Sequence
  Primer sequence Forward (5??-3??) for GAPDH
TGTTTCCTCGTCCCGTAG

SEQ ID NO: 3 - protein, 19 aa, Artificial Sequence
  Primer sequence Reverse (5??-3??) for GAPDH
CAATCTCCACTTTGCCACT

SEQ ID NO: 4 - DNA, 2347 bp, Mus musculus
aggctatctcctagttctccgctgctcggactagccattgccgccgcctcacctctgctgcaagtagcctcgccgtcggggagccctaccacagcgtccgccctcagcatgatggacttggagttgccaccgccaggactacagtcccagcaggacatggatttgattgacatcctttggaggcaagacatagatcttggagtaagtcgagaagtgtttgactttagtcagcgacagaaggactatgagctggaaaaacagaaaaaactcgaaaaggaaagacaagagcaactccagaaggaacaggagaaggccttttttgctcagtttcaactggatgaagaaacaggagaattcctcccaattcagccggcccagcacatccagacagacaccagtggatccgccagctactcccaggttgcccacattcccaaacaagatgccttgtactttgaagactgtatgcagcttttggcagagacattcccatttgtagatgaccatgagtcgcttgccctggatatccccagccacgctgaaagttcagtcttcactgcccctcatcaggcccagtccctcaatagctctctggaggcagccatgactgatttaagcagcatagagcaggacatggagcaagtttggcaggagctattttccattcccgaattacagtgtcttaataccgaaaacaagcagctggctgatactaccgctgttcccagcccagaagccacactgacagaaatggacagcaattaccatttttactcatcgatctcctcgctggaaaaagaagtgggcaactgtggtccacatttccttcatggttttgaggattctttcagcagcatcctctccactgatgatgccagccagctgacctccttagactcaaatcccaccttaaacacagattttggcgatgaattttattctgctttcatagcagagcccagtgacggtggcagcatgccttcctccgctgccatcagtcagtcactctctgaactcctggacgggactattgaaggctgtgacctgtcactgtgtaaagctttcaacccgaagcacgctgaaggcacaatggaattcaatgactctgactctggcatttcactgaacacgagtcccagccgagcgtccccagagcactccgtggagtcttccatttacggagacccaccgcctgggttcagtgactcggaaatggaggagctagatagtgcccctggaagtgtcaaacagaacggccctaaagcacagccagcacattctcctggagacacagtacagcctctgtcaccagctcaagggcacagtgctcctatgcgtgaatcccaatgtgaaaatacaacaaaaaaagaagttcccgtgagtcctggtcatcaaaaagccccattcacaaaagacaaacattcaagccgcttagaggctcatctcacacgagatgagcttagggcaaaagctctccatattccattccctgtcgaaaaaatcattaacctccctgttgatgacttcaatgaaatgatgtccaaggagcaattcaatgaagctcagctcgcattgatccgagatatacgcaggagaggtaagaataaagtcgccgcccagaactgtaggaaaaggaagctggagaacattgtcgagctggagcaagacttgggccacttaaaagacgagagagaaaaactactcagagaaaagggagaaaacgacagaaacctccatctactgaaaaggcggctcagcaccttgtatcttgaagtcttcagcatgttacgtgatgaggatggaaagccttactctcccagtgaatactctctgcagcaaaccagagatggcaatgtgttccttgttcccaaaagcaagaagccagatacaaagaaaaactaggttcgggaggatggagccttttctgagctagtgtttgttttgtactgctaaaacttcctactgtgatgtgaaatgcagaaacactttataagtaactatgcagaattatagccaaagctagtatagcaataatatgaaactttacaaagcattaaagtctcaatgttgaatcagtttcattttaactctcaagttaatttcttaggcaccatttgggagagtttctgtttaagtgtaaatactacagaacttatttatactgttctcacttgttacagtcatagacttatatgacatctggctaaaagcaaactattgaaaactaaccagaccactatacttttttatatactgtatgaacaggaaatgacatttttatattaaattgtttagctcataaaaattaaaaggagctagcactaataaaagaatatcatgacttaaa

SEQ ID NO: 5 - protein, 20 aa, Artificial Sequence
  Primer sequence Forward (5??-3??) for Nrf2
AGCAGGACATGGAGCAAGTT

SEQ ID NO: 6 - protein, 20 aa, Artificial Sequence
  Primer sequence Reverse (5??-3??) for Nrf2
TTCTTTTTCCAGCGAGGAGA

SEQ ID NO: 7 - DNA, 1552 bp, Mus musculus
aggctcagctcttactagcctagcctgtagccagccctaaggatctctccgaagagctttagggtcgtcttggcaaccagctgctcagccaatcagcgttcggtattacgatcctccctcaacatctggagccatggcggcgagaagagccctgattgtactggcccattcagagaagacatcattcaactacgccatgaaggaggctgctgtagaggctctgaagaagagaggatgggaggtactcgaatctgacctctatgctatgaacttcaaccccatcatttccagaaatgacatcacaggtgagctgaaggactcgaagaactttcagtatccttccgagtcatctctagcatataaggaaggacgcctgagcccagatattgtggccgaacacaagaagctggaagctgcagacctggtgatatttcagttcccattgcagtggtttggggtgccagccattctgaaaggctggtttgagagagtgctcgtagcaggatttgcctacacatatgctgccatgtacgacaacggtcctttccagaataagaagaccttgctttctatcaccactgggggtagcggctccatgtactctcttcagggtgtccacggggacatgaacgtcattctctggccgattcagagtggcatcctgcgtttctgtggcttccaggtcttagaacctcaactggtttacagcattggccacactccaccagatgcccgcatgcagatcctggaaggatggaagaaacgtctggaaaccgtctgggaggagaccccactctattttgctccaagcagcctgtttgacctaaactttcaggcaggattcttaatgaaaaaggaagttcaagaggagcagaagaagaacaagtttggcctctctgtgggccatcacctgggcaagtccattccagctgacaaccagatcaaagctagaaaataaggatttttttcctaacatatagttagacgcagctttctttttccccagcttgtctgacttgctttcatttttttcctttgctccacgaggatgggaaaaggagtaagtttgcttcatgctttttttttttttttgatagttctgccataacaacaaaatgaatgaagtcagattaggagcctcagggcaaggtgcagaagcgagctggaaatactcttctaggtcatttatgcaatattcgccattttcttcgggctagtcccagttagatggcatccagtcctccatcaagattcgttgtctataattacctctctgtggtttagggcagaagggaattgctcaaagtaaacaatggccgagggactaacttgtttagcagttagcagttagctaaagcctgtttatgatacatcctggtttcaattactgtgcagtgactgacatggcgcccagggggttggctctccagctcttttctgtcttgtacacagcacacccaggtcctgggaaaggaattttaaaacagatctccgtctcattctttctatttctttttttttttaatcgaaataaatgaatacatcacacatc

SEQ ID NO: 8 - protein, 19 aa, Artificial Sequence
  Primer sequence Forward (5??-3??) for NQO1
AGCCCAGATATTGTGGCCG

SEQ ID NO: 9 - protein, 20 aa, Artificial Sequence
  Primer sequence Reverse (5??-3??) for NQO1
CCTTTCAGAATGGCTGGCAC